(19) United States
(12) Patent Application Publication
(10) Pub. No.: US 2008/0269732 A1
(43) Pub. Date: Oct. 30, 2008

(54) Low Power Laser Irradiator for Treating Alopecia

(75) Inventor: Woong Beom Pyun, Gyeonggi-do (KR)

Correspondence Address: MARGER JOHNSON & MCCOLLOM, P.C., 210 SW MORRISON STREET, SUITE 400, PORTLAND, OR, 97204, US

(73) Assignee: OSTERN CO., LTD., Seoul 158-814 (KR)

(21) Appl. No.: 11/569,247

(22) PCT Filed: Mar. 15, 2005
(86) PCT No.: PCT/KR05/00746

§ 371 Date: Nov. 16, 2006

(30) Foreign Application Priority Data
May 19, 2004 (KR) 2004-0035499
Jan. 21, 2005 (KR) 2005-0005767

(51) Int. Cl. A61B 18/20

(52) U.S. Cl. 606/9

(57) ABSTRACT

A low-power laser irradiator of the present invention activates hair follicle cell and increases blood flow of scalp tissue, thus enhancing effects of hair loss prevention and hair regrowth. The laser irradiator includes a laser output unit in which red laser diodes with 630 nm˜680 nm wavelength and near-infrared laser diodes with 750 nm˜1000 nm wavelength are arranged alternatively. The comb-shaped laser irradiator is convenient to use through typical combing action and effectively used for alopecia treatment.

### TECHNICAL FIELD

The present invention relates generally to a laser irradiator and, more particularly, to a low-power laser irradiator available for curing alopecia by arranging laser diodes with different output wavelength alternatively.

Hair of human beings grows on the scalp through the growth cycle which includes three steps, i.e., catagen, telogen and anagen, and then sheds. In general, people are born with about 100,000 hairs, and each day about 80 strands of hair are shed from the scalp. Alopecia means more hair loss than grown. Alopecia may be caused by testosteron that is changed into dihydrotestosterone (DHT) under the action of 5 alpha reductase enzyme and then combined with androgen receptors. DHT may reduce the function of dermal papilla and hair follicles, resulting in hair loss. Alopecia may arise from a great variety of causes, for example, hereditary factors, hormone imbalance, emotional stress, environmental pollution, blood circulation trouble of the scalp, under-nutrition, diseases such as thyroid, autoimmunity disorder, intake of anticancer drugs or antithyroid drugs, prescription of oral contraceptive pill, and the habit of shampooing.

### BACKGROUND ART

Examples of the conventional treatment for alopecia, androgenic alopecia, a surgical operation and medical treatment have been widely used. Hair regrowth products that are currently in the market have been developed to improve the circulation of blood of the skin and to provide the nutrition to a hair root. These products, however, are not medicines, but quasi-drugs which have been not proved to be effective. Additionally, drugs that obtained the approval of FDA are only Minoxidil and Propecia. In truth, there is no excellent treatment method to satisfy alopecia patients.

Research for alopecia treatment using laser has been conducted for a long time. After the initiative research of Professor Mester in Budapest, Hungary around 1970, a stimulating effect on the biological system by low-power laser irradiation has been studied. The medical effects of low-power laser have been disclosed in many papers, for example, ┌T. I. Karu ‘Low-power laser therapy’, In: Biomedical Photonics Handbook, Ch. 48 (2003), 48-1˜48-25, Z┘, ┌M. Schaffer, H. Bonel, R. Sroka, P. M. Schaffer, M. Bosch, M. Reiser and E. Dunmke, ‘Effects of 780 nm diode laser irradiation on blood microcirculation: preliminary findings on time-dependent T1-weighted contrast-enhanced magnetic resonance (MRI)’, J. Photochem. Photibiol. B: Biol. 54 (2000) 55-60 [symbol] , [symbol] A. Schindl, G. Heinze, M. Schindl, H Pernerstorfer-Schon and L. Schindl, ‘Systemic effects of low-intensity laser irradiation on skin microcirculation in patients with diabetic microangiopathy’, Microvascular Research 64 (2002) 240˜246 [symbol] .

Moreover, technique that applies these medical effects of low-power laser to the growth of hair has been disclosed in [symbol] Dankook University Institute of Medical Laser Technology, ‘Research report on hair growing effects of 890 nm low-power laser and 680 nm LED’, 2003 [symbol] , Korean Patent Publication No. 2002-86548 entitled ‘Laser comb improved in design and function’, and International Patent Publication No. WO 02/102,228 entitled ‘Zigzag beam splitter for apparatus and method for stimulating hair growth’. The Korean Patent Publication above has disclosed a technique that laser diodes disposed in two rows of teeth generate a beam with 632 [symbol] ˜670 [symbol] visible red ray. The International Patent Publication mentioned above has disclosed a technique using a beam-splitting reflector that splits a single laser beam to ensure that energy from the laser beam is evenly distributed, and using a zigzag lens to offer multiple beams from a laser source.

### DISCLOSURE OF INVENTION

### Technical Problem

One object of the present invention is to provide a laser irradiator that is superior in treatment effect for alopecia to conventional laser therapy technology.

Another object of the present invention is to provide a laser irradiator that not only can promote the growth of hair, but also can cure alopec ia.

Still another object of the present invention is to provide a laser irradiator that is more convenient and safer to use.

### Technical Solution

A laser irradiator of the present invention includes a laser output unit in which red laser diodes with 630 mm˜680 nm wavelength and near-infrared laser diodes with 750 [symbol] ˜1000 [symbol] wavelength are arranged in turn. The laser output unit generates laser pulse having 1 [symbol] ˜5 [symbol] frequency, 100 s˜500 s pulse duration time and 1 mW˜5 mW power.

In one exemplary embodiment of the invention, four red laser diodes and three near-infrared laser diodes are arranged in turn in a row, and a plurality of comb teeth are arranged along both sides of the row of the laser diodes. Power is supplied through power distributor to the central processing unit controlling the entire operation of the laser diodes and the power output of the power distributor is selectively coupled to the central processing unit by human operator. A driving unit driving the laser diodes supplies output power of the central processing unit to a switching transistor in order to output laser in the form of pulse.

In one exemplary embodiment of the invention, switches interconnected between the power distributor and the central processing unit includes a power switch and a laser switch. When both switches are on together, laser is output.

### BRIEF DESCRIPTION OF THE DRAWINGS

FIGS. 1 to 3 are perspective views and a side view showing a laser irradiator in accordance with the present invention.

FIGS. 4 to 6 are cross-sectional views showing a comb part adapted for the laser irradiator in accordance with the present invention.

FIG. 7 is a cross-sectional view showing the arrangement of components such as a laser output unit on a circuit board used in the laser irradiator in accordance with the present invention.

FIG. 8 is a block diagram showing the circuit construction of the laser irradiator in accordance with the present invention.

FIG. 9 is a circuit diagram of the laser irradiator in accordance with one exemplary embodiment of the present invention.

FIGS. 10 to 16 are circuit diagrams available for the laser irradiator of the present invention.

FIG. 17 is a graph showing variation of average hair count according to treatment period in the first clinical trial.

FIG. 18 is a graph showing variation of hair count in silent period according to treatment period in the first clinical trial.

FIG. 19 is a graph showing variation of hair count in growth period according to treatment period in the first clinical trial.

FIG. 20 is a distribution chart of treatment effect evaluation in the first clinical trial.

FIG. 21 is a graph showing variation of average hair count according to treatment period in the second clinical trial.

FIG. 22 is a graph showing variation of hair count in silent period according to treatment period in the first clinical trial.

FIG. 23 is a graph showing variation of hair count in growth period according to treatment period in the first clinical trial.

FIG. 24 is a distribution chart of treatment effect evaluation in the first clinical trial.

### BEST MODE FOR CARRYING OUT THE INVENTION

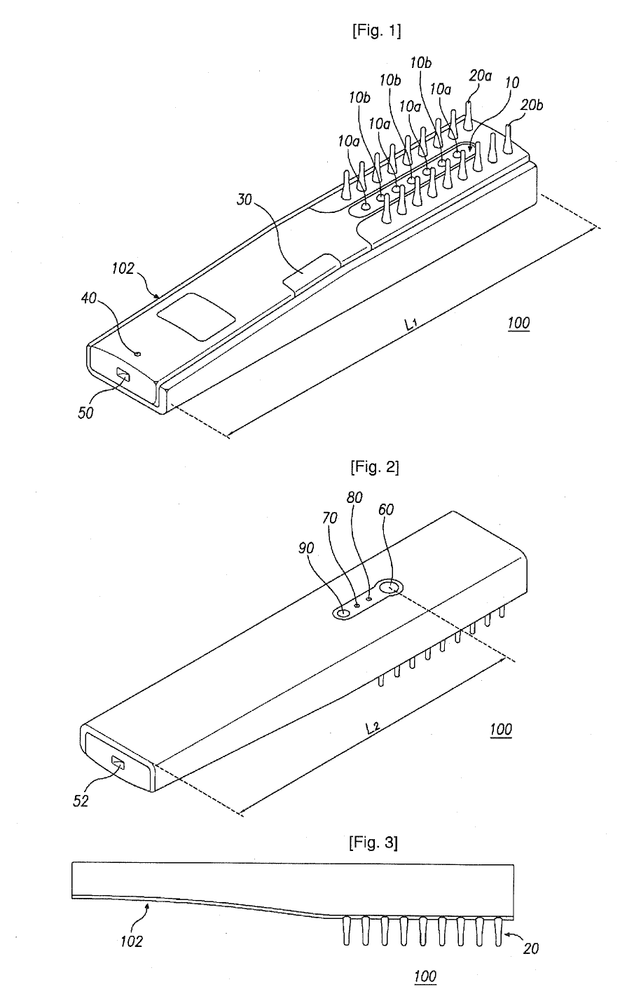

Referring to FIGS. 1˜3, a laser irradiator 100 according to the present invention includes a laser output unit 10, a first comb 20a, a second comb 20b, a laser switch button 30, a sound speaker 40, a power lead-in hole 50, a power switch button 60, a sound lamp 70, a power lamp 80, and a sound switch button 90. While a user brushes his/her hair with handle 102, the laser irradiator 100 sends forth laser to the scalp.

The laser output unit 10 has four red laser diodes 10a with 630 nm˜680 nm wavelength and three near-infrared laser diodes 10b with 750 nm˜1000 nm wavelength, which are arranged in turn in a row. Preferably, the length of the laser output unit 10 should provide irradiation range sufficient to obtain effects such as an increase in blood flow of the scalp when the laser irradiator is used in a usual combing. For example, the length of the laser output unit 10 is 60 mm. Preferably, the laser output unit 10 generates laser pulse having 1 kHz˜5 kHz frequency, 100 s˜500 s pulse duration time and 1 mW˜5 mW power.

The elements of the laser irradiator 100, such as the combs 20a and 20b, are not limited to exemplary configuration shown in FIGS. 1 to 3 and may have anther alternatives. However, the entire length of the laser irradiator 100 should be sufficient to allow a user to brush his hair with hand. Further, the power switch button 60 and the sound switch button 90 should be disposed apart from the handle 102 toward the comb part 20 for easy handling. In a preferred embodiment, the length (L1 of FIG. 1) of the laser irradiator 100 is about 230 mm, and the length (L2 of FIG. 2) to the power switch button 60 is about 125 mm.

The first and second comb 20a and 20b have a plurality of teeth that are arranged along both sides of the row of the laser output unit 10. The first and second comb 20a and 20b make hair wider, so that laser beam may easily reach the scalp. In addition, the combs 20a and 20b allow the laser output unit 10 to maintain a regular distance from the scalp.

Preferably, the laser switch button 30 can turn on the laser output unit 10 only when the power switch button 60 is on. That is, the laser output unit 10 allows emitting laser beam on condition that the laser switch button 30 is pushed after the power switch button 60 is turned on. When the laser switch button 30 is pushed again during operation of the laser output unit 10, the emission of laser beam is stopped.

The sound speaker 40 is a part to make a beep sound.

The power lead-in hole 50 may receive adapter, for example. Through the adapter, external power (for example, 220VAC, 60 Hz) is converted into direct current (for example, 7.5 VDC) to supply power necessary for operation of the laser irradiator.

The power switch button 60 supplies electric power to the laser irradiator 100, so that the emission of laser beam is ready. When the power switch button 60 is on, the power lamp 80 is turned on. The power lamp 80 consists of, for example, a red light-emitting diode (LED). Though the laser switch button 30 is pushed with the power switch button 60 off, laser beam is not emitted. If the power switch button 60 is pushed again during the emission of laser beam, the electric power is cut off and therefore, the emission of laser beam is stopped. Further, if laser emission continues for a fixed time (for example, fifteen minutes), the electric power is automatically cut off.

The sound lamp 70 is turned on when the sound switch button 90 is pushed. For example, a beep rings every eight seconds during the emission of laser beam. Further, after a lapse of fixed time (for example, ten minutes) in laser emission, a beep rings continuously three times to inform the stop time before hand. When fifteen minutes passes after use, a beep rings lengthily three times and then the emission of laser beam is stopped.

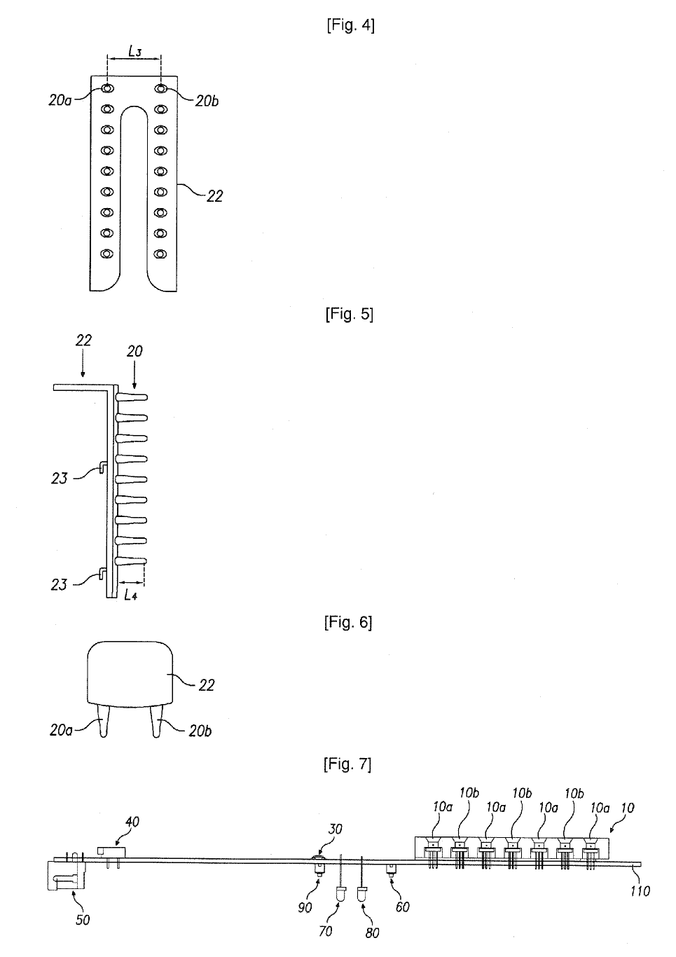

FIGS. 4 to 6 show a structure of a comb part adapted for the laser irradiator 100 of the present invention. The first comb 20a and the second comb 20b each includes nine teeth arranged in a row. In one preferred embodiment, the distance L3 between the first and second combs is, for example, 26 mm, and the length L4 of the tooth is, for example, 13.5 mm. The comb part may be fixed to a body of the laser irradiator by means of hooks 23 of a body 22 of the comb part.

On the other hand, the emission of laser beam may be made at the tip of the combs 20a and 20b by connecting the laser output unit 10 and the combs 20a and 20b with the optical fiber.

FIG. 7 is a cross-sectional view showing the arrangement of components such as the laser output unit 10 on a circuit board 110 used in the laser irradiator of the present invention. The circuit board 110 is a printed circuit board (CB), for example. As seen from the left in FIG. 7, mounted on the circuit board 110 are the power lead-in hole 50, the speaker 40, the laser switch button 30, the sound switch button 90, the sound lamp 70, the power lamp 80, the power switch button 60, and the laser output unit 10. The laser output unit 10 has a configuration in which semiconductor laser diodes different in output wavelength are alternatively arranged in a row. The semiconductor laser diode is configured to emit a laser beam at right angles to a horizontal plane of the circuit board 110. For example, as described above, the laser output unit 10 has four red laser diodes 10a with 630 nm˜680 nm wavelength and three near-infrared laser diodes 10b with 750 nm˜1000 nm wavelength, which are arranged in turn in a row.

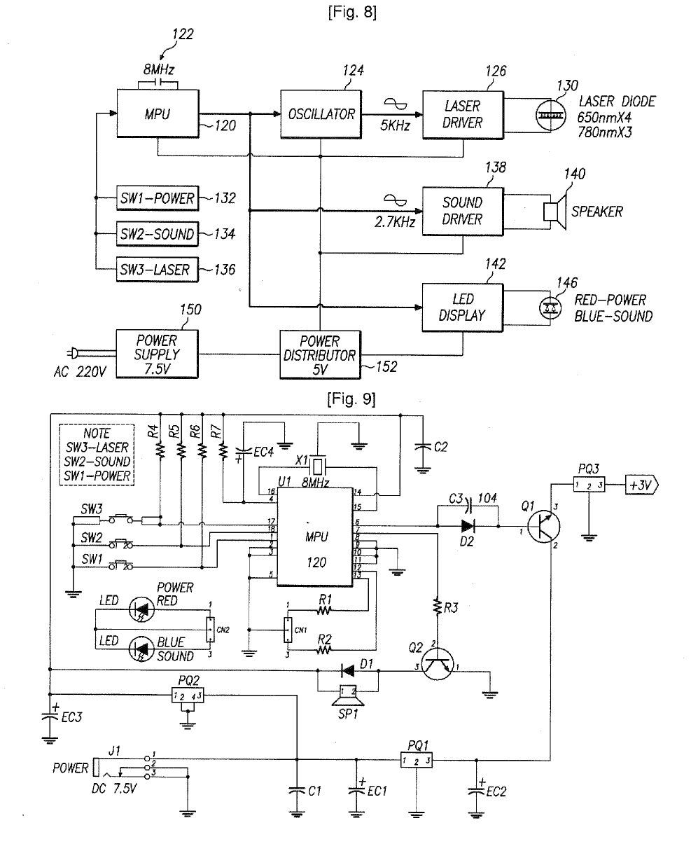

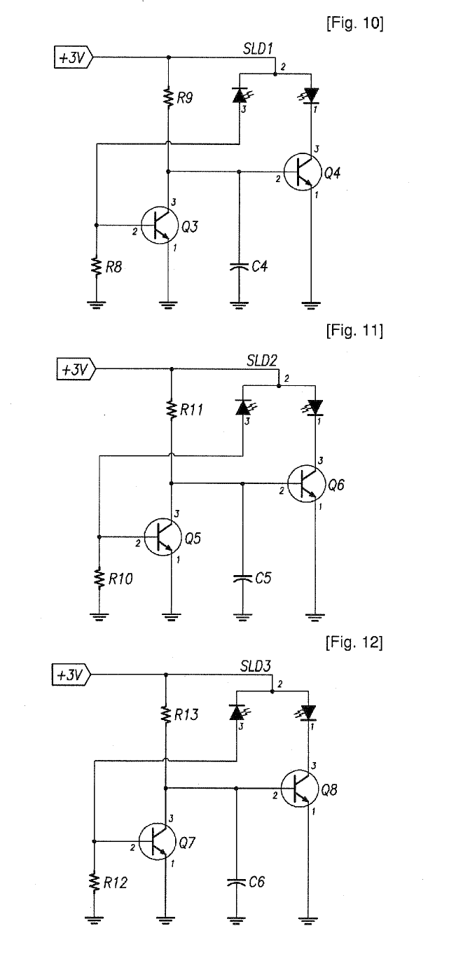

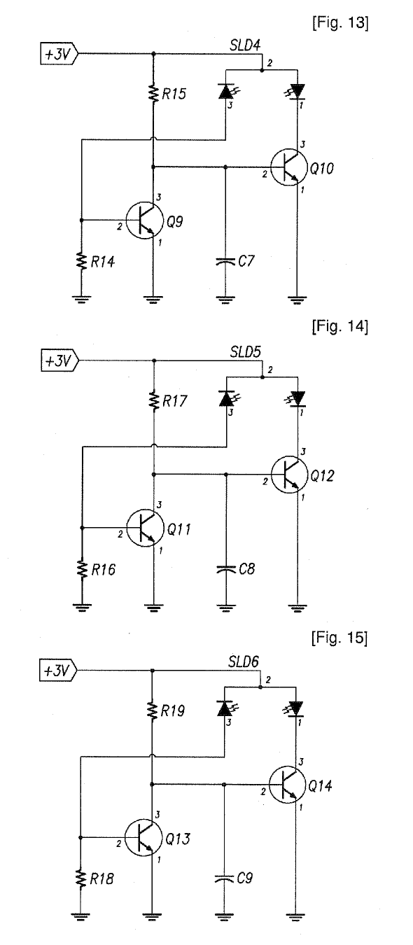

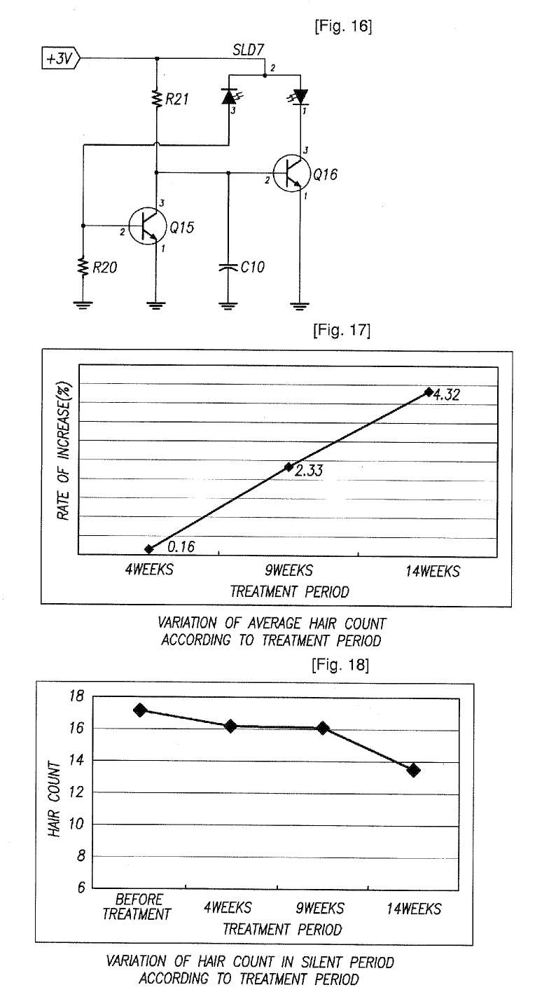

FIG. 8 is a block diagram showing the circuit construction of the laser irradiator in accordance with the present invention. FIG. 9 is a circuit diagram of the laser irradiator in accordance with one exemplary embodiment of the present invention. FIGS. 10 to 16 are circuit diagrams available for the laser irradiator of the present invention.

Referring to FIGS. 8 to 16, the laser irradiator of this invention may include a central processing unit 120 such as MPU (micro processing unit), a resonator 122, an oscillator 124, a laser driver 126, laser diodes 130, switches 132, 134 and 136, a sound driver 138, a speaker 140, an LED display 142, LEDs 146, and a power distributor 152.

The central processing unit 120 controls the entire operation of the laser irradiator, and allows the operations of the laser driver 126, the sound driver 138 and the LED display 142 according to user's button control.

7.5V/600 mA electric power into which external power (for example, AC 220V) is converted by the power supply 150 of the adapter (not shown) is supplied to the power distributor 152, which converts it into stable 5.0V/0.4 A and then applies it to the internal circuits to drive the central processing unit 120, the laser diodes 130, the speaker 140, and the LEDs 146. Depending on the actions of a power switch (SW1) 132, a sound switch (SW2) 134, and a laser switch (SW3) 136, the corresponding powers are supplied to the central processing unit 120 from the power distributor 152. The laser irradiator of this invention does not necessarily require the supply of electric power converted from external power, but may use a secondary battery such as lithium ion battery. The laser irradiator using secondary battery is handy to carry about because charging is possible and no electric cord is needed.

The resonator 122 is connected to the central processing unit 120 to produce oscillation of signals. The oscillator 124 may be built in the central processing unit 120. Among output signals of the oscillator 124, for example, 5 kHz output signal is applied to a switching transistor Q1 of the laser driver 126 so that the laser diodes (SLD1˜SLD4 and SLD5˜SLD7) 130 can emit laser in the shape of pulse having uniform wave form. Additionally, for example, 2.7 kHz output signal is applied to a switching transistor Q2 of the sound driver 138.

Red laser diodes (SLD1˜SLD4) having output wavelength of 630 nm˜680 nm and near-infrared laser diodes (SLD5˜SLD7) having output wavelength of 750 nm˜1000 nm may be configured as illustrated in FIGS. 10 to 16, for example. The red laser diode may employ InGaAlP semiconductor laser diode (for example, QL65D5SA), and the near-infrared laser diode may employ AlGaAs semiconductor laser diode (for example, QL78C6SA).

The laser diodes fabricated according to this invention were tested. The accuracy of laser emission was measured at the tip of the laser output unit 10 by using a laser power-meter. As a result of the test, the red laser 10a showed the maximum output of 2.3 mW 0.9%, and the near-infrared laser 10b did the maximum output of 1.5 mW 2.0%, both of which satisfied the acceptable error range, i.e., 20%. Further, the stability [(P1−P2)/P1 100] of laser emission was examined by measuring a first output P1 in five minutes and a second output P2 in ten minutes after starting operation. The red laser 10a and the near-infrared laser 10b showed good results, 1.3%, 2.0%, respectively. Further, a result of measuring the area of laser emitted at the tip of comb teeth was 80 mm in width and 9 mm in length (error 1%). The wavelength at the maximum laser intensity, measured by laser wavelength analyzer, was 655 nm 6.4 nm in the red laser and 780 nm 5.2 nm in the near-infrared laser. The frequency of laser pulse measured by oscilloscope was 5 kHz 0.8%. The pulse duration time of output laser pulse, obtained by Full Width Half Maximum (FWHM) after measurement of laser wavelength analyzer, was 70 nm 4.5 nm in the red laser and 60 nm 4.0 nm in the near-infrared laser.

It should be noted that the laser irradiator of the present invention is not limited to the construction shown in FIGS. 8 to 16.

Clinical Trial for Effectiveness Evaluation

To evaluate the effectiveness of the low-power laser irradiator for hair loss prevention, a clinical trial was carried out. Testee for the clinical trial were selected according to the following.

Acceptable testee: (1) androgenic alopecia patient more than twenty years of age, (2) androgenic alopecia patient with II-V grades in Norwood-Hamilton classification, (3) man who maintains uniform hair style during the clinical trial period, (4) patient who does not dye or care for hair during the clinical trial period, and (5) patient who signs a written consent.

Non-acceptable testee: (1) patient who takes or applies adrenocortical hormone drugs, (2) patient who uses antiandrogenic drugs within six months before the clinical trial, (3) patient who uses finasteride within six months before the clinical trial, (4) patient who uses minoxidil or the like within six months before the clinical trial, (5) patient who has another types of alopecia except androgenic alopecia, and (6) patient who is judged inadequate due to other reasons.

Testee used the low-power laser irradiator of the invention once a day, five times a week, ten minutes in a time. The period in use was fourteen weeks, and there was no combined treatment. Testee used and kept the low-power laser irradiator of the invention, as follows.

(1) Preparation points before use: (i) full knowledge of how to use, (ii) removal of dirt or dust.

(2) How to use: (i) to power the laser irradiator, (ii) to push the laser switch button after directing laser emission path to the scalp, (iii) to emit laser at one spot of the scalp, to move the laser irradiator about 2 cm after eight seconds, and to emit laser again, (iv) to repeat the above (iii) step until laser is emitted to the entire scalp, (v) to power off.

(3) How to keep after use: (i) to power off, (ii) to clean a surface of the laser irradiator and then to put it into a box, (iii) to keep the box at a safe place free from vibration, shock, and moisture.

The procedure of the clinical trial was as follows.

(1) to select testee according to standards for acceptance and non-acceptance.

(2) to instruct testee in related information such as the object and the procedure of the clinical trial, and the usage of the laser irradiator.

(3) A person in charge of the clinical trial executes the follow items before the start of the clinical trial and after the selection of testee.

\textcircled{1} to record the grade of testee in Norwood-Hamilton classification on a record document.

\textcircled{2} Global photography: to take a photograph of an upper part of the head, to attach the photograph onto the record document, and to record photographing conditions (camera, focus, distance, flash, etc). Testee should wash his hair to remove gloss on hair.

\textcircled{3} Phototrichogram: to select respectively target areas among hair-loss parts where androgenic alopecia proceeds and non-hair-loss parts in order to measure hair count per unit area. Next, to cut hair with 1 mm in each target area with circular area of 1 [symbol] , and to indicate the central point by means of tattooing. A CCD microscope is used to take an enlarged photo of the target area. An image analyzer captures photo image and indicates the circular target area as an imaginary line in a computer. By marking the position of hair in the target area, this is expressed as user's selected color, and hair count is automatically measured. Hair count is then recorded to the record document, and a printed photo image is attached to the record document.

(4) Testee starts treatment using the laser irradiator of the invention five times a week, once a day, ten to fifteen minutes in a time.

(5) Testee answers questions about a degree of satisfaction in treatment at fourth, ninth and fourteenth weeks from the start of treatment. Further, hair count per unit area is measured and recorded. During fourteenth week, a person in charge of the clinical trial executes again the above steps \textcircled{2} and \textcircled{3}, then synthetically judges treatment effect, and records.

(6) After the clinical trial is finished to all testee, testing results are reported to the committee.

Result 1 of the Clinical Trial

Twenty-six testee took part in the clinical trial which the Applicant executed in Hanyang University Hospital. The age of testee ranged from twenty-six to sixty. No adverse reaction happened in treatment period.

The result of observation on variation of average hair count during treatment period of fourteen weeks is shown in the following Table 1 and FIG. 17.

TABLE 1

| | | | variation of | |
|---|---|---|---|---|
| | treatment | average hair | total hair count | rate of increase |
| part | period | count (/□) | (Δ) | (%) |
| hair loss | before | 117.5 | — | — |
| | treatment | | | |
| | 4th week | 118.0 | 0.54 | 0.16 |
| | 9th week | 122.0 | 3.43 | 233 |
| | 14th week | 122.7 | 5.15 | 432 |

As shown in Table 1 and FIG. 17, hair count in hair loss part after treatment increases by 4.32% (p<0.005) in comparison with the treatment before, and this means statistically significant effect on a prevention against alopecia. Specifically, there is no statistical significance on hair loss prevention until fourth week. However, a significance is found on the ninth week, and a relatively high significance is obtained at the final fourteenth week.

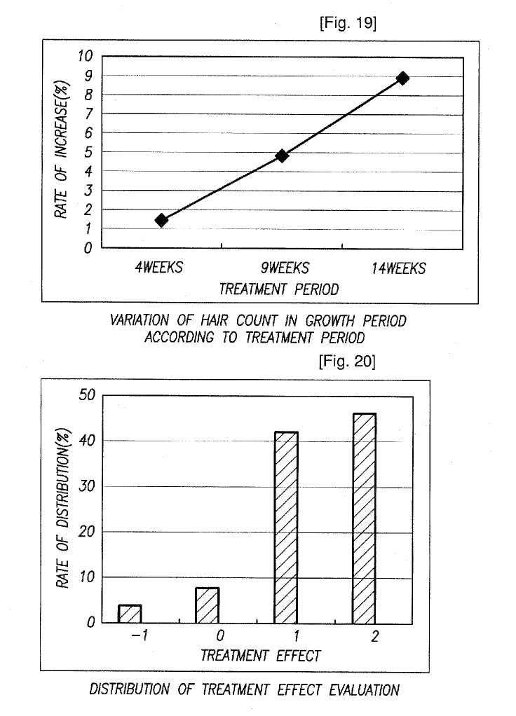

With the progress of hair loss, hair count in silent period increases by degrees, but hair count in growth period decreases. Therefore, variations of hair count in both periods were observed to study effectiveness of effect on hair loss prevention. Variation of hair count in silent period is shown in the following Table 2 and FIG. 18. Variation of hair count in growth period is shown in the following Table 3 and FIG. 19.

As shown in Tables 2, 3 and FIGS. 17, 18, at fourth week, hair count in silent period decreases by 3.84% (p>0.05), and hair count in growth period increases by 1.42% (p>0.05). This is a result without statistical significance. However, from ninth week, a decrease of hair count in silent period and an increase of hair count in growth period are statistically significant.

TABLE 2

| | | | variation of hair | |
|---|---|---|---|---|
| | treatment | average hair | count in silent | rate of decrease |
| part | period | count (/□) | period (Δ_{T}) | (%) |
| hair loss | before | 17.2 | — | — |
| | treatment | | | |
| | 4th week | 16.2 | −1.00 | 3.84 |
| | 9th week | 16.1 | −1.83 | 8.27 |
| | 14th week | 13.5 | −3.65 | 21.9 |

TABLE 3

| | | | variation of hair | |
|---|---|---|---|---|
| | treatment | average hair | count in growth | rate of increase |
| part | period | count (/□) | period (Δ_{A}) | (%) |
| hair loss | before | 100.4 | — | — |
| | treatment | | | |
| | 4th week | 101.9 | 1.54 | 1.42 |
| | 9th week | 106.0 | 5.26 | 4.81 |
| | 14th week | 109.2 | 8.81 | 8.92 |

Synthetically considering a variation of hair count after the clinical trial, variations of hair count in growth period and silent period, and so forth, a person in charge of the clinical trial judged treatment effect. Such evaluation grades are shown in the following Table 4, and a distribution thereof is shown in FIG. 20. As seen from Table 4 and FIG. 20, testee who got grade 1 or more occupied 88.5%. Accordingly, the laser irradiator of the present invention seems to have an effect on androgenic alopecia.

TABLE 4

| | |
|---|---|
| evaluation grade of | |
| treatment effect | distribution (26 persons) |

| | | |
|---|---|---|
| −1 (worse) | 1 | 3.8% |
| 0 (no effect) | 2 | 7.7% |
| 1 (effective) | 11 | 42.3% |
| 2 (remarkably effective) | 12 | 46.2% |

Result 2 of the Clinical Trial

Twenty-four testee took part in the second clinical trial which the Applicant executed in Kangdong Seongsim Hospital. The age of testee ranged from twenty-seven to fifty-seven. No adverse reaction happened in treatment period.

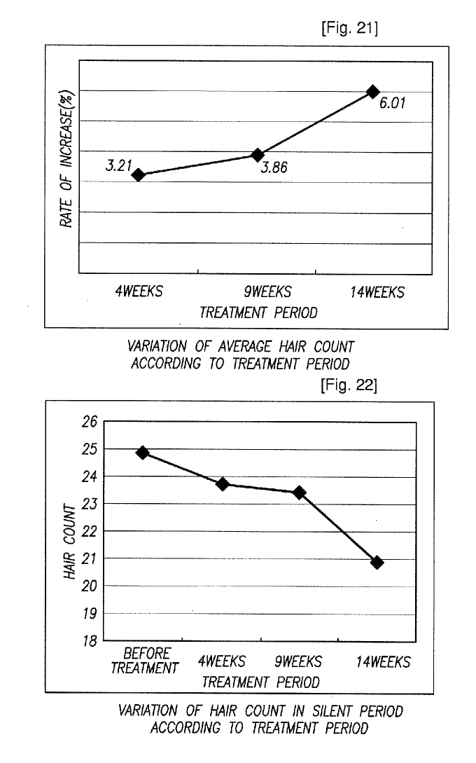

The result of observing variation of average hair count during treatment period of fourteen weeks is shown in the following Table 5 and FIG. 21.

TABLE 5

| | | | variation of | |
|---|---|---|---|---|
| | treatment | average hair | total hair count | rate of increase |
| part | period | count (/□) | (Δ) | (%) |
| hair loss | before | 153.3 | — | — |
| | treatment | | | |
| | 4th week | 157.9 | 4.63 | 3.21 |
| | 9th week | 159.4 | 6.21 | 3.86 |
| | 14th week | 163.3 | 10.1 | 6.01 |

As shown in Table 5 and FIG. 21, hair count in hair loss part after treatment increases by 6.01% (p<0.005) in comparison with before treatment, and this means statistically significant effect on a prevention against alopecia.

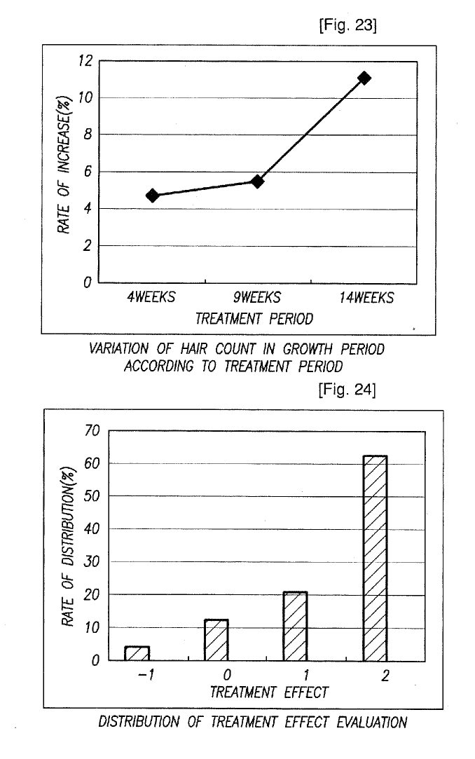

Variation of hair count in silent period is shown in the following Table 6 and FIG. 22. Variation of hair count in growth period is shown in the following Table 7 and FIG. 23. As shown in Tables 6, 7 and FIGS. 22, 23, from fourth week, hair count in silent period decreases by 4.52% (p<0.05), and hair count in growth period increases by 4.72% (p<0.005). From this, a decrease of hair count in silent period and an increase of hair count in growth period are statistically significant.

TABLE 6

| | | | variation of hair | |
|---|---|---|---|---|
| | treatment | average hair | count in silent | rate of decrease |
| part | period | count (/□) | period (Δ_{T}) | (%) |
| hair loss | before | 24.8 | — | — |
| | treatment | | | |
| | 4th week | 23.7 | −1.2 | 4.52 |
| | 9th week | 23.4 | −1.4 | 6.94 |
| | 14th week | 20.9 | −3.9 | 17.4 |

TABLE 7

| | | | variation of hair | |
|---|---|---|---|---|
| | treatment | average hair | count in growth | rate of increase |
| part | period | count (/□) | period(Δ_{A}) | (%) |
| hair loss | before | 128.4 | — | — |
| | treatment | | | |
| | 4th week | 134.2 | 5.8 | 4.72 |
| | 9th week | 136.0 | 7.6 | 5.52 |
| | 14th week | 142.4 | 14.0 | 11.1 |

Synthetically considering on variation of hair count after the clinical trial, variations of hair count in growth period and silent period, and so forth, a person in charge of the clinical trial judged treatment effect. Such evaluation grades are shown in the following Table 8, and a distribution thereof is shown in FIG. 24. As seen from Table 8 and FIG. 24, testee who got grade 1 or more occupied 83.3%. Accordingly, the laser irradiator of the present invention seems to have an effect on androgenic alopecia.

TABLE 8

| | |
|---|---|
| evaluation grade of | |
| treatment effect | distribution (26 persons) |

| | | |
|---|---|---|
| −1 (worse) | 1 | 4.2% |
| 0 (no effect) | 3 | 12.5% |
| 1 (effective) | 5 | 20.8% |
| 2 (remarkably effective) | 15 | 62.5% |

### INDUSTRIAL APPLICABILITY

Since emitting a coherent light in a straight line, contrary to a light-emitting diode, the laser irradiator according to the present invention penetrates the outer layer of the skin and transmits effectively photo energy to the inner layer of the skin. Moreover, the laser irradiator of the present invention employs laser diodes emitting horizontally an oval-shaped light, and further, uses two types of laser diodes with different output wavelength. Therefore, the laser irradiator of this invention is superior in hair loss prevention effect and hair regrowth effect to conventional laser irradiator using laser diodes with single output wavelength or using both laser diode and light-emitting diode.

Particularly, the laser irradiator of the present invention applies laser output to the scalp by alternatively arranging red laser diodes with 630 nm˜680 nm wavelength and near-infrared laser diodes with 750 nm˜1000 nm wavelength, so the hair follicle cell can be activated and blood flow of the scalp tissue can be increased. The laser irradiator of the invention can increase the amount of blood flow more than 50% without greatly changing the temperature of the scalp. Increased blood flow not only provides much nourishment and oxygen to the scalp, but also transfers testosteron of high density accumulated in the scalp tissue to another portion. This results in treatment of alopecia and cleaning effect similar with washing hair. In addition, combined laser emitted from the laser irradiator of the invention not only promotes the formation of hair follicle located in the inner layer of the skin, but also stimulates the follicle cell so that hair root is much more secured.

When laser output is in the form of pulse having 1 kHz˜5 kHz frequency, 100 s˜500 pulse duration time and 1 mW˜5 mW low-power, the activation of the follicle cell and the increase of blood flow can be maximized.

Furthermore, because of assuming a comb shape, the laser irradiator of the invention is convenient to use through typical combing action.

Besides, the laser irradiator permits laser output by means of twice operation, and the laser output is automatically stopped when a fixed time passes after use. Generation of a beep during laser emission and one-touch stop of laser emission can provide much safer laser irradiator.

1. A low-power laser irradiator comprising:
a laser output unit including a first laser diode having a first output wavelength and a second laser diode having a second output wavelength, wherein the first output wavelength is different from the second output wavelength, and wherein the first and second laser diodes are arranged alternatively; and
a comb part brushing user's hair when laser emitted from the laser output unit is applied to user's scalp.

2. The low-power laser irradiator of claim 1, wherein the first wavelength is 630 nm˜680 nm, and the second wavelength is 750 nm˜1000 nm.

3. The low-power laser irradiator of claim 1 or claim 2, wherein the laser output unit generates a laser pulse having 1 kHz˜5 kHz frequency.

4. The low-power laser irradiator of claim 3, wherein the laser pulse has 100 s˜500 pulse duration time.

5. The low-power laser irradiator of claim 1 or claim 2, wherein the laser output unit generates a laser pulse having 1 mW˜5 mW power.

6. A low-power laser irradiator comprising:
a laser output unit including a first laser diode having 630 nm˜680 nm wavelength and a second laser diode having 750 nm˜1000 nm wavelength, the first and second laser diode being arranged alternatively; and
a comb part brushing user's hair when laser emitted from the laser output unit is applied to user's scalp,
wherein the laser output unit generates laser pulse having 1 kHz˜5 kHz frequency, 100 s˜500 s pulse duration time and 1 mW˜5 mW power.

7. The low-power laser irradiator of claim 1 or claim 6, wherein the laser output unit includes four first laser diode having 630 nm˜680 nm wavelength and three second laser diode having 750 nm˜1000 nm wavelength, the first and second laser diode being arranged in turn in a row, and the comb part includes a plurality of comb teeth being arranged along both sides of the row of the laser diodes.

8. The low-power laser irradiator of claim 1 or claim 6, further comprising:
a central processing unit controlling the entire operation of the laser irradiator;
a power distributor converting power necessary to operation of the laser irradiator and supplying the converted power to the central processing unit;
a switch selectively connecting power output of the power distributor to the central processing unit according to user's operation; and
a laser driver driving the laser diodes of the laser output unit according to output signals of the central processing unit.

9. The low-power laser irradiator of claim 8, wherein the central processing unit applies oscillating output to a switching transistor of the laser driver.

10. The low-power laser irradiator of claim 8, wherein the switch includes a power switch and a laser switch, and wherein the laser output unit emits laser only when both of the power switch and the laser switch are on.

11. The low-power laser irradiator of claim 10, wherein the switch further includes a sound switch, and wherein a beep generates when the sound switch is on.

12. A low-power laser irradiator comprising:
a laser output unit including a first laser diode with 655 nm wavelength and a second laser diode with 780 nm wavelength, the first and second laser diode being arranged alternatively;
a comb part brushing user's hair when laser emitted from the laser output unit is applied to user's scalp;
a central processing unit controlling the entire operation of the laser irradiator;
a power distributor converting power necessary to operation of the laser irradiator and supplying the converted power to the central processing unit;
a switch selectively connecting power output of the power distributor to the central processing unit according to user's operation; and
a laser driver driving the laser diodes of the laser output unit according to output signals of the central processing unit,
wherein the laser output unit generates laser pulse having 1 kHz˜5 kHz frequency, 100 s˜500 s pulse duration time and 1 mW˜5 mW power.
